(19) United States
(12) Patent Application Publication
(10) Pub. No.: US 2023/0201113 A1
(43) Pub. Date: Jun. 29, 2023

(54) IMMUNE BOOSTER - SUPPLEMENT TREATMENT KIT AND METHODS OF USE

(71) Applicant: Altered Labs LLC, Fort Lauderdale, FL (US)

(72) Inventor: Dario ECHEVERRY CAMPOS, Miramar, FL (US)

(21) Appl. No.: 17/998,694

(22) PCT Filed: May 14, 2021
(86) PCT No.: PCT/US2021/032556

§ 371 Date: Nov. 14, 2022

Related U.S. Application Data
(60) Provisional application No. 63/025,685, filed on May 15, 2020.

(51) Int. Cl. A61K 9/00; A61K 9/08; A61K 31/4706; A61K 31/395; A61P 31/14; A61K 47/34; A61K 9/46; A23L 33/16; A23L 33/105

(52) U.S. Cl. CPC A61K 9/0095; A61K 9/08; A61K 9/0043; A61K 31/4706; A61K 31/395; A61P 31/14; A61K 47/34; A61K 9/0007; A23L 33/16; A23L 33/105

(57) ABSTRACT

A method of preventing viral infection and/or treating viral infection and/or one or more symptoms of viral infection is disclosed. Also disclosed are compositions prepared using reducing gas which are useful for treating viral infection or symptoms thereof and/or preventing viral infection. Various dosage forms prepared using said compositions are described, including drinkable formulations, concentrated drops, concentrated syrups, compositions formulated for nasal administration, and tablets and capsules. A kit for preparing said compositions is also described.

### CROSS-REFERENCE TO RELATED APPLICATIONS

This application is an international patent application filed under the Patent Cooperation Treaty and claims priority to U.S. Provisional Patent Application No. 63/025,685, filed on May 15, 2020, the entire content of which is incorporated by reference herein.

### BACKGROUND OF THE INVENTION

### Technical Field

The present invention is directed to compositions, a treatment kit and methods of use thereof to prevent and/or treat viral infections in humans and/or one or more symptoms thereof.

Covid-19 was first detected in Wuhan City, Hubei Province, China in 2019, and that has now been detected in many locations internationally, including cases in the United States. The virus has been named “SARS¬ CoV-2” and the disease it causes has been named “Coronavirus Disease 2019” (COVID-19). On Jan. 31, 2020, the Department of Health and Human Services (HHS) issued a declaration of a public health emergency related to COVID-19 and mobilized the Operating Divisions of HHS.1 In addition, on Mar. 13, 2020, the President declared a national emergency in response to COVID-19.2 (FDA, 2020).

In humans, coronaviruses mostly cause respiratory and gastrointestinal symptoms. Clinical manifestations range from a common cold to more severe disease such as bronchitis, pneumonia, severe acute respiratory distress syndrome, multi-organ failure and even death. Coronavirus deaths are linked to patients' immune systems that have an inflammatory response to the virus causing Acute Respiratory Distress Syndrome (ARDS). With ARDS, the entire lung is affected, unlike pneumonia where often only part of the lung is affected (Zimmermann & Nigel, 2020).

Oxidative stress is considered to be part of the pathogenic mechanism for lung infections and pneumonia and is closely linked to inflammation; i.e., (1) attenuation of oxidative stress has been found to reduce pulmonary damage; and antioxidants have been found to be effective in alleviating lung injury and protecting against damage of other organs (Qianwen et al., 2018); (2) Oxidative stress takes part in host innate immune response to foreign pathogens, and increases the production of mediators of pulmonary inflammation (Akkaya et al., 2008); (3) The effects of oxidative stress in the airway as well as in other organs depend on Ratio of Oxidative Stress (ROS) concentration and time of exposure. In general, higher levels of ROS produce damage in biomolecules (e.g., lipid peroxidation) and induce intracellular signaling pathways leading to cell death, mainly through apoptosis (Valko et al., 2007).

Oxidative stress is considered to be part of the pathogenic mechanism for lung infections and pneumonia and is closely linked to inflammation. In particular, attenuation of oxidative stress has been found to reduce pulmonary damage; and antioxidants have been found to be effective in alleviating lung injury and protecting against damage of other organs. Oxidative stress takes part in host innate immune response to foreign pathogens, and increases the production of mediators of pulmonary inflammation.

Coronavirus-related deaths have been found to be linked to patients' immune systems that have an inflammatory response to the virus, thereby causing Acute Respiratory Distress Syndrome (ARDS). During respiratory burst, there is a rapid release of reactive oxygen species (ROS). Further investigation into the mechanism of SARS-CoV-2 indicates that it attacks the endothelium forming the interior of blood and lymph vessels in the body, resulting in ARDS in compromised elderly patients, but also strokes in younger patients. Thus, it can be concluded that SARS¬ CoV-2 is an endothelial disease using the lungs to enter the body.

Oxidative stress has been shown to be a key factor in SARS¬ CoV-2 infection. (See Roche and Mesta, Archives of Medical Research, April 2020.) The mechanism of action for this virus inhibits key enzymes causing further oxidative stress and in many cases death. The virus has been shown to inactivate the enzyme Angiotensin-Converting enzyme 2 (ACE-2), an enzyme attached to the cell membranes of cells in the lungs, arteries, heart, kidney, and intestines. ACE-2 normally converts the hormone Angiotensin 2 into Angiotensin 1,7 and since NAPDH Oxidase is not inhibited by Angiotensin 1,7, superoxide ion concentration increases, leading to oxidative stress. The effects of oxidative stress in the airway as well as in other organs depend on ROS concentration and time of exposure. In general, higher levels of ROS produce damage in biomolecules (e.g., lipid peroxidation) and induce intracellular signaling pathways leading to cell death, mainly through apoptosis.

In particular, SARS-CoV-2 recruits polymorphonuclear neutrophils (PMNs) which use NADPH Oxidase to further produce superoxide ions. The buildup of superoxide ions leads to oxidative stress and, without enough free electrons, the electron cascade in the mitochondria shuts down and ATP production is reduced, leading to apoptosis and ultimately cell death.

Additionally and more generally, certain viral infections have been associated with the redox modifications characteristic of oxidative stress. Alteration of the endogenous levels of glutathione (GSH), e.g., has been found in experimental infections in vitro with herpes simplex virus type 1 (HSV-1), Sendai virus, HIV and in vivo with influenza A virus and HSV-1. GSH levels are decreased in plasma, peripheral blood mononuclear cells and monocytes in asymptomatic HIV infected individuals and in AIDS patients.

There is thus an ongoing need for new antiviral treatments, including prophylactic and remedial treatments. Additionally and relatedly, there is also an ongoing need for new methods of strengthening (“boosting”) the human immune system as either a supplement or alternative to vaccination. In particular, there is also a need for therapeutic formulations which do not introduce harmful chemicals into recipients' bodies and which do not elicit adverse reactions or produce undesired side effects.

Through extensive experimentation, the present inventor has developed a novel method for converting liquids, including infused liquids, into highly effective anti-oxidative compositions (i.e., solutions). In particular, the method of the present invention involves an electrolytic process for producing a non-toxic, non-corrosive, stable, reducing gas that can be infused into water/liquids. The electrolytic process, also termed “Hydrogras™” reduces the liquid oxidation reduction potential (ORP), and increases dissolved free electrons, as well as hydroxide (OH⁻) and free hydrogen (H₂) content.

ORP is the measure of free oxygen and/or other oxidizing agents present in a liquid, such as water. The determination of ORP is generally significant in water which contains a relatively high concentration of a redox-active species, e.g., the salts of many metals (such as Fe²⁺, Fe³⁺) and strong oxidizing agents (such as chlorine) and reducing agents (such as sulfite ions). ORP is measured in millivolts (mV) and the more oxygen that is present in the water, the higher the ORP measurement.

The present invention reduces oxidative stress in individuals infected with a virus by providing to the body free electrons through an anti-oxidative solution taken as an immune booster treatment to combat any oxidative stress caused.

The inventive method utilizes a sodium silicate complex which is a silicon-based alkaline solution having a highly basic pH. The complex's elemental and chemical properties give it unique electrochemical and structural characteristics that appear to be directly related to the different ways the complex regulates redox processes. The complex's multiple ionizable forms give it the ability to accept and donate electrons and participate in important redox reactions. The compound is obtained in a series of specific reactions involving a gamut of different liquid sodium silicate complexes. In preferred embodiments, the sodium silicate complex used is sodium metasilicate, which is an approved food additive and has been granted GRAS status by the FDA.

The method of the present invention further utilizes glutathione, which is a complex that stabilizes glutathione in a reduced form and that can be further delivered through any non-hairy area of the skin. The complex protects glutathione from oxidation, thereby preserving glutathione's antioxidant properties until it enters a recipient subject. Glutathione and the complex has been granted GRAS status by the FDA.

### SUMMARY

The present disclosure provides for a method of treating or preventing a viral infection or a symptom thereof comprising administering to an individual in need thereof a composition comprising an aqueous solution, wherein the composition is prepared by a process comprising infusing the aqueous solution with a reducing gas and a metasilicate, wherein the reducing gas and/or the metasilicate reacts with the aqueous solution to produce a reducing liquid having an oxidation reduction potential (ORP) value of about −100 mV or more negative.

The present disclosure further provides for a composition for preventing or treating viral infection or symptoms thereof, comprising an aqueous solution infused with a metasilicate and reducing gas, wherein the ORP value of the composition is −100 mV or more negative.

Other aspects of the present invention will be made apparent by the following detailed description. Additional aspects of the present invention will be readily apparent to a person of ordinary skill in the art in view of the following disclosure.

### DETAILED DESCRIPTION

Set forth below is a detailed description of a method for preparing compositions described herein useful for treating viral infections, a method for treating viral infections comprising administering to a person in need thereof a composition described herein, and compositions useful for treating viral infections, all representing examples of the inventions disclosed here.

The detailed description set forth below is intended as a description of various configurations of the subject technology and is not intended to represent the only configurations in which the subject technology may be practiced. The detailed description includes specific details for the purpose of providing a thorough understanding of the subject technology. However, it will be apparent to those skilled in the art that the subject technology may be practiced without these specific details. In some instances, well-known structures and components are shown in block diagram form in order to avoid obscuring the concepts of the subject technology. Like components are labeled with identical element numbers for ease of understanding.

It is understood that various configurations of the subject technology will become readily apparent to those skilled in the art from the disclosure, wherein various configurations of the subject technology are shown and described by way of illustration. As will be realized, the subject technology is capable of other and different configurations and its several details are capable of modification in various other respects, all without departing from the scope of the subject technology. Accordingly, the summary and detailed description are to be regarded as illustrative in nature and not as restrictive.

Unless otherwise defined, all technical and scientific terms used herein have the same meaning as commonly understood by a person having ordinary skill in the art to which the present invention belongs. While methods and materials similar or equivalent to those described herein can be used to practice the invention, suitable methods and materials are described herein. All publications, patent applications, patents, and/or other references mentioned herein are incorporated by reference in their entireties. In the event that any of the publications, patent applications, patents and/or other references mentioned and incorporated herein contradict the present disclosure, the present disclosure including the definitions is authoritative. Additionally, the materials, methods, and examples are illustrative only and are not intended to be limiting.

The exemplary methods disclosed herein are based on the combination of a highly reducing, negatively charged gas such as “Hydrogas™”, and a highly reducing, high alkaline liquid sodium metasilicate (RLS). The RLS according to the present invention may be formed with any high alkaline, non-caustic, human-grade (e.g., food grade) liquid. The highly reducing gas may also be any highly reducing, negatively charged gas, including but not limited to such gases as HHO, BROWNS Gas, Tylar Gas, Knell Gas, etc.

Additional enhancers have also been utilized in exemplary embodiments of the method that combine with the above mentioned products, mostly being anti-oxidant, non-acidic, non-reactive products including but not limited to: natural honey, natural ginger roots, sodium saccharin, alkaline fruit juices, etc.

The doses and protocols described herein are exemplary only, and the dosages, treatment protocol, and means of administration may vary in other exemplary uses of the methods.

The articles “a” and “an” are used herein to refer to one or to more than one (i.e. to at least one) of the grammatical object of the article. By way of example, an element means one element or more than one element.

As used herein in reference to a value, the term “about” refers to a value that is similar, in context to the referenced value. In general, those skilled in the art, familiar with the context, will appreciate the relevant degree of variance encompassed by “about” in that context. For example, in some embodiments, the term “about” can encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value. The details of one or more embodiments of the invention are set forth in the description below. Further features, objects and advantages of the invention will become apparent from the description as well as from the claims.

Unless otherwise indicated, all numbers expressing quantities of ingredients, properties such as molecular weight, reaction conditions, and so forth used in the present specification and associated claims are to be understood as being modified in all instances by the term “about.” Accordingly, unless indicated to the contrary, the numerical parameters set forth in the following specification and attached claims are approximations that may vary depending upon the desired properties sought to be obtained by the embodiments of the present disclosure. At the very least, and not as an attempt to limit the application of the doctrine of equivalents to the scope of the claim, each numerical parameter should at least be construed in light of the number of reported significant digits and by applying ordinary rounding techniques.

Whenever a numerical range of degree or measurement with a lower limit and an upper limit is disclosed, any number and any range falling within the range is also intended to be specifically disclosed. For example, every range of values (in the form “from a to b,” or “from about a to about b,” or “from about a to b,” “from approximately a to b,” and any similar expressions, where “a” and “b” represent numerical values of degree or measurement) is to be understood to set forth every number and range encompassed within the broader range of values.

All numerical ranges defined herein are inclusive of endpoints and all values thereinbetween, unless otherwise specifically stated. For example, “at a concentration of a-b” means “at a concentration of at least a and at most b.”

As used herein, the terms “subject” and “recipient” refer to human and non-human animals, including veterinary subjects. The term “non-human animal” includes all vertebrates, e.g., mammals and non-mammals, such as non-human primates, mice, rabbits, sheep, dog, cat, horse, cow, chickens, amphibians, and reptiles. In a preferred embodiment, the subject is a human.

As used herein, the term “administration” refers to the administration of a composition to a subject or system, for example to achieve delivery of said composition and/or a therapeutic agent which is included in, or is otherwise delivered by, the composition.

As used herein, the term “agent” refers to a substance, entity or complex, combination, mixture or system, or phenomenon (e.g., heat, electric current or field, magnetic force or field, etc.). For example, a flavoring agent is a substance imparting flavor to a composition.

As used herein, “amelioration” refers to the prevention, reduction or palliation of a state, or improvement of the state of a subject. Amelioration includes, but does not require complete recovery or complete prevention of a disease, disorder or condition (e.g., radiation injury).

As used herein, “associated with” denotes a relationship between two events, entities and/or phenomena. Two events, entities and/or phenomena are “associated” with one another, as that term is used herein, if the presence, level and/or form of one is correlated with that of the other. For example, a particular entity is considered to be associated with a particular disease, disorder, or condition, if its presence, level and/or form correlates with incidence of and/or susceptibility to the disease, disorder, or condition (e.g., across a relevant population).

Those skilled in the art will appreciate that the term “composition”, as used herein, can be used to refer to a discrete physical entity that comprises one or more specified components. In general, unless otherwise specified, a composition can be of any form, e.g., gas, gel, liquid, solid, etc.

As used herein, the terms “pharmaceutically acceptable” or “therapeutically acceptable” as applied to any carrier, diluent, or other additive or excipient used to formulate a composition as disclosed herein means that the carrier, diluent, additive or other excipient is compatible with the other ingredients contained in the composition and is not deleterious to the recipient thereof. Similarly, as used herein, the term “pharmaceutically acceptable carrier” or “therapeutically acceptable carrier” means a pharmaceutically or therapeutically material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

As used herein when used in connection with the occurrence of a disease, disorder, and/or condition, “prevent” (and grammatical variations thereof) refers to reducing the risk of developing the disease, disorder and/or condition and/or to delaying onset of one or more characteristics or symptoms of the disease, disorder or condition. Prevention can be considered complete when onset of a disease, disorder or condition has been delayed for a predefined period of time.

An individual who is “susceptible to” a disease, disorder, or condition is at risk for developing the disease, disorder, or condition. In some embodiments, an individual who is susceptible to a disease, disorder, or condition does not display any symptoms of the disease, disorder, or condition. In some embodiments, an individual who is susceptible to a disease, disorder, or condition has not been diagnosed with the disease, disorder, and/or condition. In some embodiments, an individual who is susceptible to a disease, disorder, or condition is an individual who has been exposed to conditions associated with development of the disease, disorder, or condition. In some embodiments, a risk of developing a disease, disorder, and/or condition is a population-based risk (e.g., family members of individuals suffering from the disease, disorder, or condition).

As used herein, the term “subject” or “patient” refers to an organism, typically a mammal (e.g., a human). In some embodiments, a subject is suffering from a relevant disease, disorder or condition. In some embodiments, a subject is susceptible to a disease, disorder, or condition. In some embodiments, a subject displays one or more symptoms or characteristics of a disease, disorder or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is an individual to whom diagnosis and/or therapy is and/or has been administered.

As used herein, the term “therapeutically effective amount” refers to an amount that produces the desired effect for which it is administered. In some embodiments, the term refers to an amount that is sufficient, when administered to a population suffering from or susceptible to a disease, disorder, and/or condition in accordance with a therapeutic dosing regimen, to treat the disease, disorder, and/or condition. In some embodiments, a therapeutically effective amount is one that reduces the incidence and/or severity of, and/or delays onset of, one or more symptoms of the disease, disorder, and/or condition. Those of ordinary skill in the art will appreciate that the term “therapeutically effective amount” does not in fact require successful treatment be achieved in a particular individual. Rather, a therapeutically effective amount can be that amount that provides a particular desired pharmacological response in a significant number of subjects when administered to patients in need of such treatment. In some embodiments, reference to a therapeutically effective amount can be a reference to an amount as measured in one or more specific tissues (e.g., a tissue affected by the disease, disorder or condition) or fluids (e.g., blood, saliva, serum, sweat, tears, urine, etc.). Those of ordinary skill in the art will appreciate that, in some embodiments, a therapeutically effective amount of a particular agent or therapy can be formulated and/or administered in a single dose. In some embodiments, a therapeutically effective agent can be formulated and/or administered in a plurality of doses, for example, as part of a dosing regimen.

As used herein, the term “treatment” (and grammatical variations thereof) refers to administration of a therapy that partially or completely alleviates, ameliorates, relieves, inhibits, delays onset of, reduces severity of, and/or reduces incidence of one or more symptoms, features, and/or causes of a particular disease, disorder, and/or condition, or is administered for the purpose of achieving any such result. In some embodiments, such treatment can be of a subject who does not exhibit signs of the relevant disease, disorder and/or condition and/or of a subject who exhibits only early signs of the disease, disorder, and/or condition. Alternatively or additionally, such treatment can be of a subject who exhibits one or more established signs of the relevant disease, disorder and/or condition. In some embodiments, treatment can be of a subject who has been diagnosed as suffering from the relevant disease, disorder, and/or condition. In some embodiments, treatment can be of a subject known to have one or more susceptibility factors that are statistically correlated with increased risk of development of the relevant disease, disorder, and/or condition. In various examples, treatment is of a cancer. Tumor: As used herein, the term “tumor” refers to an abnormal growth of cells or tissue. In some embodiments, a tumor can comprise cells that are precancerous (e.g., benign), malignant, pre-metastatic, metastatic, and/or non-metastatic. In some embodiments, a tumor is associated with, or is a manifestation of, a cancer. In some embodiments, a tumor can be a disperse tumor or a liquid tumor. In some embodiments, a tumor can be a solid tumor.

As used herein, “Hydrogas™” refers to a reducing gas prepared according to the electrolytic process described in the present disclosure.

As used herein, “restructuring” refers to a process for transforming a liquid into a reducing liquid. As used herein, “restructured liquid” or “reducing liquid” refers to a liquid which has undergone restructuring.

As used herein, the terms “infuse” or “infusion” or “infusing” or any variation thereof encompasses any other suitable method of mixing reducing gas or silicate with liquid, such as injecting, administering, or applying. In some embodiments, a process is provided for preparing a stable, non-toxic, non-corrosive reducing liquid by infusing a gas produced by the electrolytic process described herein into a “source liquid” to be treated using described processes. The source liquid can be any suitable liquid that can stably incorporate an infused reducing gas. Examples of suitable source liquids include, but are not limited to, organic solvents, nonpolar oils, mineral oils, essential oils, colloidal suspensions, colloidal solutions, leachates from landfills, polychlorinated byphenols (PCBs), and aqueous compositions. In preferred embodiments, the source liquid for infusion is water to be used to prepare cell culture media. Sources of water include for example, distilled water, deionized water, tap water, potable water, potable beverages, nonpotable water, agricultural water, irrigation water, salt water, brackish water, fracking waters, water having aqueous heavy metals dissolved therein, industrial water, recycled water, fresh water, water from a natural source, or reverse osmosis water. Potable water is understood to be water safe for human or animal consumption; non-potable water is not safe for human or animal consumption, but can be used in other applications. Fresh water is understood to be water from a natural source that is not salt water. Salt water may be from a natural source such a sea or ocean, it also includes man-made salt water. Industrial water is water that is a used in industrial applications such as manufacturing processes, washing of containers, machines, etc. Industrial water may be tap water, well water, etc that is typically non-potable water.

As used herein, the term “substantially free” refers to quantities of less than about 1%, preferably less than about 0.1% for the indicated matter.

In an aspect, the present invention involves restructuring a liquid in, such as water or an aqueous solution, to a reducing liquid to be subsequently used to prepare a therapeutically effective composition. The liquid restructuring is performed using a non-toxic and stable reducing gas to decrease the amount of undesirable oxidants (e.g., ROS) present in the liquid.

The process for preparing a reducing gas may comprise preparing an activator, wherein the activator comprises water, potassium hydrate, magnesium sulfate, sodium oxidanide, and an alkaline metal silicate; introducing the activator into a reaction chamber of a reactor, wherein the reactor is configured to produce an electrolytic reaction; adding water to the reaction chamber to provide a water-activator mixture; and applying a direct current in the water-activator mixture to produce the reducing gas. It is generally desirable that the pressure in the reaction chamber is reduced to increase the rate of production of the reducing gas. In a preferred embodiment, the reducing pressure in the reaction chamber is maintained at about 0.5 bar. The reactor chamber typically comprises a wet electrolytic cell to propel the electrolytic reduction process as described herein. Additional information may be found in WO2019/232387, the relevant disclosures of which are incorporated by references for the subject matter and purpose referenced herein.

The activator may be prepared using any suitable equipment for conducting chemical reactions involving the activator reagents. Typically, the activator is prepared by combining the activator components in a balanced stoichiometric amounts from the oxidation-reduction equation. In some embodiments, the activator comprises potassium hydrate, magnesium sulfate, sodium oxidanide, and an alkaline metal silicate in a predetermined stoichiometric ratio. The activator can comprise about 40 wt % to about 59 wt % potassium hydrate; about 0.1 wt % to about 5 wt % magnesium sulfate; about 40 wt % to about 59 wt % sodium oxidanide; and about 0.1% to about 5 wt % alkaline metal silicate. In other embodiments, the activator can comprise about 45 wt % to about 55 wt % potassium hydrate; about 0.2 wt % to about 3 wt % magnesium sulfate; about 45 wt % to about 55 wt % sodium oxidanide; and about 0.2% to about 3 wt % alkaline metal silicate. In other embodiments, the activator can comprise about 47 wt % to about 53 wt % potassium hydrate; about 0.2 wt % to about 1.5 wt % magnesium sulfate; about 47 wt % to about 53 wt % sodium oxidanide; and about 0.2% to about 1.5 wt % alkaline metal silicate. In other embodiments, the activator can comprise about 48 wt % to about 51 wt % potassium hydrate; about 0.3 wt % to about 0.8 wt % magnesium sulfate; about 48 wt % to about 51 wt % sodium oxidanide; and about 0.3% to about 0.8 wt % alkaline metal silicate. Potassium hydrate, magnesium sulfate, and sodium oxidanide are commercially available. In other embodiments, the activator is a liquid solution comprising potassium hydrate, magnesium sulfate, sodium oxidanide, and an alkaline metal silicate in any of the stoichiometric amounts described herein. The liquid solution can have an activator concentration of about 0.1 to about 20 g/l, about 0.1 to about 15 g/l, about 0.1 to about 10 g/l, about 0.1 to about 5 g/l, about 0.5 to about 4 g/l, about 0.5 to about 3 g/l, about 1 to about 3 g/l, or about 1.5 to about 2.5 g/l.

The activator can be prepared by any suitable method. For example, the potassium hydrate, sodium oxidanide, alkaline cationic silicate, and magnesium sulfate can be measured out in any of the weight ratios described herein, and subsequently combined to form a single activator mixture. This activator mixture can then be dissolved into water at a predetermined concentration as described hereinabove. Alternatively, a quantity of water can be provided, and the potassium hydrate, sodium oxidanide, alkaline cationic silicate, and magnesium sulfate can be added to the quantity of water in sequence, simultaneously, or combined pairs. In some embodiments, the magnesium sulfate and the alkaline cationic silicate are first mixed into the quantity of water, and the potassium hydrate and sodium oxidanide are subsequently mixed into the quantity of water. Preparation of the activator can be carried out external to a reactor and subsequently added in. Alternatively, the activator can be prepared in a reaction chamber of a reactor. Preferably, the alkaline cationic silicate is a metasilicate such as an alkaline sodium silicate complex (SSC) or reformed liquid silica (RLS). The metasilicate can be used in the preparation of an activator, and may optionally be added in greater quantities with or without the reducing gas into the source liquid. These complexes are described, for example, in US 20110059189A1, which is incorporated herein by reference. Mass spectroscopic (MS) and nuclear magnetic resonance (NMR) analysis generated a putative empirical formula of the compound or complex to be Na8.2Si4.4H9.70i7.6. The formula suggests that alkaline sodium silicate complex (SSC) is not a single compound but a mixture of two different compounds that are in equilibrium with each other. Specifically, the SSC is a mixture of trimeric sodium silicate (Na₂SiO₃)₃, Na Na⁴ Na⁴:

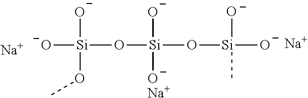

and Sodium Silicate Pentahydrate (Na₂Si0₃) 5H₂O.

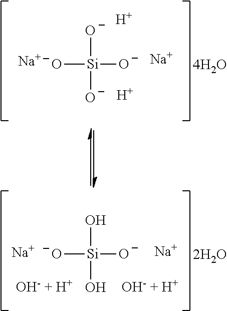

Sodium silicate pentahydrate (Na₂Si0₃) 5H2O typically exists in equilibrium as two structural forms, with one form containing one ionized water molecule and the other form containing 3 ionized water molecules. To produce SSC, silicon metal (any grade) is loaded into a reactor. Sodium oxidanide is added along with water. An exothermic reaction occurs. The reaction is allowed to proceed for 4-6 hours, after which the product is collected in a cooling tank. The product is cooled and the obtained liquid product is packaged.

The silicon-based alkaline composition (empirical formula of Na8.2Si4.4H₉₇0i7.6) can have a specific density in the range of 1.24 to 1.26 kg/m³, for example, 1.25±0.1 kg/m³. The composition can also have a pH in the range of 13.8 to 14.0, for example, 13.9±0.1. In some embodiments, the SSC can be dried via any suitable method prior to use in any of the processes described herein. Suitable drying methods include, but are not limited to, mild heating, storage in a desiccator, vacuum drying.

SSC physiochemical properties and potential therapeutic applications have been previously studied. In one study, SSC was found to exhibit antimicrobial properties for gram positive, gram negative, and drug resistant strains as described, for example, in Vatten et al., Res. J. Microbiol. 2012 Mar. 1; 7(3): 191-8. Sodium silicate is also generally recognized as safe for human consumption by the US FDA pursuant to 21 C.F.R. § 182.90. US 20140087003A1 describes a method using an alkaline sodium silicate composition to inhibit the toxic effects of venom and treat venomous bites and stings. US 20060275505A1 describes a composition for increasing alkalinity in the body containing water, a source of alkalinity; particularly an alkaline silicon solution. US20110059189A1 describes a modified sodium silicate composition, and methods of treating cancer and viral infections utilizing the modified sodium silicate composition (Na82Si44H₉70 i76), also described in Townsend et al., Int. J. Appl. Res. Nat. Prod. 2010; 3:19-28 (AVAH silicates were also effective in inhibiting several important physiological events important in survival and development of virulence in viral and microbial pathogens). However, the SSC referenced in those publications did not involve a reducing gas, the combination of which is a subject under this description, along with other beneficial uses of this technology.

The electrolytic process is generally carried out in a reactor. In an exemplary process, the activator is either prepared within a reaction chamber of the reactor or externally prepared and subsequently added to the reaction chamber. Additional water can be combined with the activator in the reaction chamber in any suitable quantity, including up to the fill capacity of the reaction chamber.

The reactor can be any suitable apparatus for carrying out an electrolytic reaction. In some embodiments, the reactor comprises a wet electrolytic cell. In an electrolytic cell, an electric current is passed from an electronic conductor through a chemical substrate such as an ionic solution contained in one or more cells (i.e., reaction chamber), back into a second electronic conductor. The circuit is closed outside (external circuit) of the cell through various electronic conductors. This typically includes a power supply and a current measuring device. The junctions between the electronic and ionic conductors are called electrodes, namely cathodes and anodes. In the electrolysis reaction, a direct current is passed through the solution contained in the reaction chamber, producing chemical reactions at the electrodes. In a standard electrolysis of pure water (i.e., without activator present), a reduction half reaction occurs at the cathode in which electrons from the cathode are transferred to hydrogen cations to form H₂ gas as illustrated by the chemical equation: 2H+(aq)+2e H₂(g). At the anode, an oxidation half reaction occurs in which electrons are transferred from water molecules to the anode to form 0₂ gas as illustrated by the chemical equation: 2H₂0(1) 0₂(g)+4H⁺ (aq)+4e−. These half reactions can be balanced with the addition of base.

A direct current (DC) electrical supply is coupled to the reactor and provides the energy necessary to drive the electrolytic process. Electric current is carried by electrons in the external circuit. Electrodes of metal, graphite and semiconductor material are widely used. Choice of suitable electrode depends on chemical reactivity between the electrode and electrolyte and manufacturing cost. A DC electrical power source is connected to two electrodes, or two plates (typically made from some inert metal such as platinum, stainless steel 360 or iridium) which are placed in the water. In some embodiments, the DC delivered to the electrolytic cell is in the range of about 20 V to about 30 V, for example about 24.65 V±0.12 V. The input of electrical current can be further be through a 110 V (60 Hz) or 220 V, 50 Hz or 60 Hz circuit.

The reactor can be configured to perform the electrolytic reaction under reduced pressure or in a vacuum. Vacuum-electrolysis reactors are known in the art and suitable apparatuses will be readily apparent to a person of ordinary skill. The electrolysis reaction can be conducted at standard temperature and pressure (STP). In some embodiments, the reaction is initially conducted at STP, then subsequently, once the production of reducing gas begins inside the reactor chamber, the pressure can be reduced inside the reaction chamber. For example, the reduced pressure can be about 0.3 bar to about 0.9 bar. In some embodiments, the reduced pressure is 0.5±0.05 bar. By performing the reaction under reduced pressure, the rate of production of the reducing gas can be increased by up to 2.2 fold over the reaction performed at standard atmospheric pressure.

In some embodiments, the liquid can be an aqueous solution having medium to high biochemical oxygen demand (BOD). BOD is defined as the amount of dissolved oxygen needed by aerobic biological organisms to break down organic material present in a given water sample, most commonly expressed in milligrams of oxygen consumed per liter of sample during 5 days of incubation at 20° C. In some embodiments, the aqueous solution has a 5-day BOD in the range of about 2 mg/F to about 600 mg/F.

Infusion can be conducted by any suitable method. For example, the gas can be infused into the liquid by bubbling the reducing gas into the liquid. The bubbling can be conducted simultaneously with electrolytic production of the reducing gas by coupling the reactor to a container having the liquid therein and flowing the reducing gas into the liquid as it is produced. Alternatively, the infusion can be conducted by bubbling a stored reducing gas, such as in a pressurized gas tank, into a container having the liquid therein.

The infusion process can be augmented by adding the reducing gas to the liquid under turbulent conditions. In fluid dynamics, turbulence or turbulent flow is any pattern of fluid motion characterized by chaotic changes in pressure and flow velocity. Turbulence is caused by excessive kinetic energy in parts of a fluid flow, which overcomes the damping effect of the fluid's viscosity. In general terms, in turbulent flow, unsteady vortices appear of many sizes which interact with each other. Turbulent conditions can be created by a variety of methods that are well-known, which include, but are not limited to, vortexing, shaking, vibrating, mixing, flotation, and cavitation. Turbulence and cavitation improve dissolution rate of the reducing gas into the liquid by up to 100-fold, depending on the application and on the flow capacity of the recirculating pump, typically measured in volume units (e.g. gallons, liters) per minute. In some embodiments, the turbulent conditions are produced by cavitation, wherein the cavitation is conducted using a propeller, impeller, or suitable device. In one example, a recirculating pump is used that contains an impeller, at a rate of up to 3600 revolutions per minute (RPM), preferably 750-900 RPM. Venturi technology is also used when the turbulence is created inside pipes that have a positive flow pressure of liquids.

In producing the stable reducing liquid, the reducing gas is infused into the liquid until a threshold negative ORP is achieved and observed for a sufficient amount of time (stabilization or retention time) to reliably measure the ORP value using a commercially available and calibrated ORP meter with a waterproof electrode, preferably one that can also measure pH. A person of ordinary skill in the art will understand the routine conventions associated with the measurement of reduction potentials, including standard oxidation reduction potentials. This stabilization time will vary depending on the amount of liquid produced per unit of time. In some embodiments, the stabilization time is at least about 2 minutes. In other embodiments, the stabilization time is at least about 10 minutes. More generally, the stabilization time will vary from a few seconds to 28 hours, depending on several factors including the degree of chemical oxygen demand (COD) and the presence or absence of colloidal particulates, oils, solvents and/or others dissolved solutions. Reduced pressure and turbulence will improve the efficiency and thus will reduce the retention time by up to a factor of 100. Appropriate methods for the determination of the appropriate stabilization time for a liquid sample of interest are within the technical knowhow of a person of ordinary skill in the art. The induction of reduced pressure and turbulence will also allow the generation of a “residual effect” in many cases. For example, by applying the correct stabilization time, the infused liquid will maintain a reducing and disinfecting residual effect (i.e. replacing oxidants like chlorine, ozone, UV, H2O2, etc). In some embodiments, the threshold ORP after stabilization is −150 mV or more negative.

A composite reducing liquid comprising a nontoxic, non-corrosive reducing agent and the infused reducing liquid described herein can also be prepared. The nontoxic, non-corrosive reducing agent can be any compound that is readily miscible with the infused reducing liquid. Suitable reducing agents include, but are not limited to, natural antioxidants for example, ascorbic acid (vitamin c), glutathione, melatonin, and water-soluble tocopherols (vitamin E). In some embodiments, the non-toxic, non-corrosive reducing agent is an alkaline cationic silicate as described herein. The composite reducing liquid can be produced by any suitable method. In some embodiments, the non-toxic, non-corrosive reducing agent is added in a predetermined quantity to an infused reducing liquid. In other embodiments, the reducing agent and the reducing gas are simultaneously infused into a liquid. This simultaneous infusion can be conducted under turbulent conditions, such as using a recirculating pump at a rate of at least about 800±35 RPM.

The addition can be conducted by quantitative transfer of a single aliquot into the infused reducing liquid. Alternatively, the addition can be conducted by a continuous transfer of the reducing agent from a storage vessel at any desired flow rate over a specific period of time. The flow rate(s) and time will depend on the reducing agent and the desired stoichiometric ratio of reducing agent to infused reducing liquid in the composite reducing liquid. In another embodiment, the reducing agent is added in a punctuated, drop-wise fashion comprising multiple aliquots.

In some embodiments of the process for producing an aqueous reducing liquid, the infusion step of reducing gas, previously described, is performed by infusing 75 to 120 liters per minute of reducing gas per every 60 gallons per minute of the liquid to be restructured, prior to or simultaneously with the alkaline cationic silicate in the range of 0.5 to 12 milligrams per liter. In other embodiments, the quantity of the alkaline cationic silicate required in the process step is in amounts described herein-above, wherein the alkaline cationic silicate comprising of lithium silicate, sodium silicate, potassium silicate, ammonium silicate, or a combination thereof.

In one aspect, the process for preparing a reducing liquid comprising infusing a reducing gas (e.g. a reducing gas produced by an electrolytic process described herein) into a quantity of liquid under turbulent conditions. Inducing turbulence and cavitation in this process increases the efficiency of restructuring the water in the tank up to a thousand fold. It allows for the use of 1 kw of power per every ten thousand (10,000) gallons of water to be restructured per hour. Without the implementation of the cavitation/turbulence system, the rate of dissolution of gas with liquid is inefficient for utility. However, the upper limit for turbulent conditions in this process is less than 3600 RPM because excessive turbulence leads potential cavitation of the impeller of the water pump, which is undesirable for utility.

In some embodiments, the restructuring process comprises the following steps: reducing water gas (“C1”) and reducing liquid metasilicate (“C2”) are injected immediately before the source liquid enters into any conventional reservoir or container. The source liquid to be treated may go through (i) a closed pressured pipe; or (ii) an open water tank, channel, or open pipe under atmospheric conditions or normal temperature and pressure conditions.

If the source liquid to be treated goes through a closed pressurized pipe, the following steps are further performed: (i) C1 and C2 are injected to the pipe, where C1 is injected via a Venturi apparatus or via another method of creating negative pressure in the pipe; (ii) C2 is proportionally injected via conventional dosing pumps, gravitational dosing methods, or any other method used to dosify liquid chemicals. Negative pressure improves the production of the liquid. Depending on the electrolytic cell, the improvement of gas production can be up to 250%. Different tests conducted show with accuracy that it takes about 9325 liters of C1 gas under NPT conditions to restructure, in about 10 hours, 5000 gallons of water to be treated. This value is equivalent to 932.5 liters of C1 per hour without using enhancing methods of cavitation. The flow of reducing gas (C1) is then measured as flow in liters per hour (FLPH) using a formula that varies depending on the source liquid and other parameters, described further herein for each source liquid and corresponding use. Once the closed pressurized system is stabilized, The ORP value is measured in millivolts (mv). The ORP will vary depending on the composition of the source liquid. The minimum contact time of C1 with the source liquid required inside the pipe is typically between 3 seconds and 30 minutes. The ORP charge is measured after at least 3 seconds of minimum contact time of C1 with the source liquid and should result in a negative value. The formula for calculating FLPH is irrelevant of the liquid pressure inside the pressurized pipe. The volume (milliliters) of liquid metasilicate (C2) required to restructure a source liquid (C2) is determined using a formula described herein-below, which varies based on the composition of the source liquid and its desired use.

If the liquid to be treated goes through atmospheric pressure (open tank, channel or open pipe) or under normal temperature or pressure conditions, then following steps apply for mixing C1 and C2: (i) C1 is mixed with source liquid via under turbulent conditions or via cavitation induced by using flotation modes, recirculating pumps creating vacuum and/or a Venturi apparatus; (ii) C2 is mixed with the source liquid via existing conventional dosing pumps, gravitation dosifiers, or analogous methods apparent to a person with ordinary skill in the art. The FLPH of C1 is in then measured in liters per hour using a formula specific that varies based on the composition of the source liquid and process conditions, described further herein-below which varies based on the composition of the source liquid, process conditions, and the desired use for the source liquid. The volume (milliliters) of liquid metasilicate required to restructure water (C2) is determined using a formula described herein-below, which also varies based on the composition of the source liquid, process conditions, and the desired use for the source liquid. The minimum contact of C2 in the source liquid reservoir or container is typically between 15-30 minutes to achieve a negative ORP. If the residual negative ORP value (mv) is less than −200 mV, then contact time is extended until the ORP is more negative than −200 mV.

One aspect and specific application of the restructuring process is to prepare potable or “ready to drink” (RTD) water or other beverages for human and nonhuman (animals) consumption. The restructuring process described herein-above can be applied to any water based product suitable for human and nonhuman consumption including but not limited to drinking water, carbonated beverages, juices, colored beverages, organic beverages, teas, coffees, energy drinks, CBD beverages containing cannabinoid oil, and any other beverage with added organic and/or inorganic chemical components. Wherein, the reduced drinking water is (1) substantially free of alkaline chemicals, such as but not limited to, sodium or potassium hydroxide or sodium bicarbonate; and (2) substantially free of oxidants, such as but not limited to, calcium hypochlorite, sodium hypochlorite, gaseous chlorine, bromine, iodine, ozone, or ultraviolet light. An additional benefit of the reducing or restructuring process is that the original color, taste, and odor of the reducing drinking water is preserved. Substantially free refers to oxidant quantities less than about 1%, preferably less than about 0.1% for the indicated matter.

Under normal conditions of preservation and handling, the hydration (surface tension) and alkalinity (pH) stay stable for at least 12 months after the restructuring process. Stability studies were conducted adding 1.0 mg/liter of SSC to (i) a 55 gallon drum made of BPA plastic human grade (with zero UV penetration); (ii) 1 L metallic bottles; (iii) 1 L glass bottles; (iv) 1 L plastic bottles. The reducing gas was infused into each container with a contact time of 30 minutes. Post stabilization, the pH was measured to be around 10. The drum and bottles were sealed was then kept outside under atmospheric conditions for two years in Florida, USA. After two years, the pH of the water bottle was still around 10, without any microbial growth.

The stability of the liquid water is increased because the reducing water is substantially free of oxidants because they are effectively neutralized via the reduction process, particularly oxidants such as of calcium hypochlorite, sodium hypochlorite, gaseous chlorine, bromine, iodine, ozone, and/or ultra violet light. The thus restructured water may then be used to prepare a cell culture medium of the present invention.

In some embodiments, the reducing liquid is restructured water or restructured aqueous solution.

In some embodiments, the reducing liquid obtained has a pH of about 7, or 7-14, or 7-13, or 7-12, or 7-11, or 7-10, or 7-9, or 7-8, or 8-14, or 8-13, or 8-12, or 8-11, or 8-10, or 8-9, or 9-14, or 9-13, or 9-12, or 9-11, or 9-10, or 10-14, or 10-13, or 10-12, or 10-11, or 11-14, or 11-13, or 11-12, or 12-14, or 12-13, or 13-14.

In other embodiments, the obtained reducing liquid has a pH of at least about 7.0, or at least about 9.5, or at least about 13.0.

Further, after undergoing the restructuring process, despite an alkaline pH of over 9.5 measured as equivalent oxidation reduction potential (ORP) greater than (−300 my), the resulting solution is nonetheless non-caustic, and non-toxic to humans and animals upon contact or ingestion, including an even highly alkaline pH of over 13.0 with ORP value greater than (−550 my).

The addition/infusion of the liquid metasilicate is not chemically induced, nor produced by alkaline chemicals (such as sodium hydroxide, sodium bicarbonate, etc).

Furthermore, the disinfecting and bactericidal properties inherent to reducing restructured water enhances the storage and shelf life of therapeutic compositions prepared therewith.

In an aspect, the restructuring described herein lowers the ORP value of a liquid.

In some embodiments, the restructuring converts the ORP from a positive to a negative value. Decreasing the ORP charge to a negative value is desirable because it alleviates the oxidative stress of a system, which is known in the art to be harmful to a particular system.

In other embodiments, a composition of the present invention has an ORP value of −50 mV or more negative, or −100 mV or more negative, or −200 mV or more negative, or −300 mV or more negative, or −400 mV or more negative, or about −50 mV to about −800 mV, or about −400 mV to about −600 mV, preferably about −300 mV to about −500 mV, more preferably about −200 mV to about −400 mV. In some embodiments, the composition has an ORP value of −800 mV or even more negative.

Further, compared to the non-restructured form of the same liquid, the restructured form of the liquid will exhibit additional properties, for example, a pH greater than 7, decreased surface tension, improved hydration, improved bio-assimilation, improved solubility of organic or inorganic compounds with the liquid (such as growth factors or other factors or additives necessary for or beneficial to cell culturing), improved detoxification/flush of cells, and improved cellular synthesis.

The present inventors have found that the electrolytic process described herein releases free electrical charge via the water-based reducing gas and the liquid metasilicate and its reducing, high alkaline, non-caustic, and nontoxic properties. Compositions described herein prepared by the electrolytic process described herein are useful for treating viral infections and/or symptoms thereof, and/or for preventing viral infection.

In an exemplary application of the method, positive results have been maximized by creating a “treatment kit” combining Hydrogas™ and the RLS, into one of the following formats and used according to the corresponding treatment protocols described below.

An exemplary non-limiting treatment kit of the present invention comprises a sodium silicate complex, i.e. a silicon-based alkaline solution of pH of 13.7. The elemental and chemical properties of the silicon-based alkaline solution give it unique electrochemical and structural characteristics that the present inventors have found to be directly related to the different ways of regulating redox processes (i.e., its multiple ionizable forms), which confer it the ability to accept and donate electrons and participate in important redox reactions. The sodium silicate complex is obtained in a series of specific reactions involving a gamut of different liquid sodium silicate complexes. Sodium metasilicate is an approved food additive and has been granted Generally Recognized as Safe (GRAS) status as food supplements by the United States Food and Drug Administration.

“Hydrogas™” is an electrolytic process for the production of a non-toxic, non-corrosive, stable, reducing gas that can be infused into liquids, including water and aqueous solutions, including liquids intended for human consumption (e.g., drinking or intravenous administration). The electrolytic process reduces the liquid oxidation reduction potential and increases dissolved free electrons, as well as Hydroxide (OH⁻) and free H₂ content.

Glutathione (Glutaril™) is a complex that stabilizes glutathione in a reduced form and that can be further delivered through any non-hairy area of the skin. The complex protects from oxidation of glutathione hence, preserving the antioxidant properties until it reaches the body. Glutathione and the complex have been granted Generally Recognized as Safe (GRAS) status as food supplements by United States Food and Drug Administration.

In exemplary uses according to the present invention, combinations of the above compounds turn infused liquids into highly effective anti-oxidative solutions with therapeutic properties.

Drinkable Formula

In a first embodiment, the present disclosure provides for compositions prepared using a reducing liquid described herein, which is a drinkable solution (a “drinkable formula”).

In certain embodiments of the method of treatment according to the present invention, a subject who is infected with a virus (tested positive) drinks about 10-100 mL of the drinkable formula once, twice, three times, or four times a day.

In some embodiments, an infected subject drinks about 20-80 mL of the drinkable formula three or four times a day. In other embodiments, an infected subject drinks about 30-70 mL of the drinkable formula three or four times a day.

In certain embodiments, the drinkable formula may optionally contain one or more flavorants or palatants. Inclusion of one or more flavoring agent may aid in subject compliance. Said flavorants or palatants may be one or more natural or artificial flavoring agent. For example and without limitation, compositions described herein may contain honey, ginger, turmeric, matcha powder or other powdered tea, one or more extracts (e.g., vanilla) one or more sugars or sweeteners (e.g., sucrose, fructose, sodium saccharin, sucralose), one or more fruit juices such as alkaline fruit juices, one or more vegetable juices, or other flavoring agents which a skilled person may select.

In certain embodiments, the drinkable formula may be mixed with another liquid, such as milk, coffee, tea, juice, and the like.

In an aspect, an infected subject continues drinking the drinkable formula for as long as symptoms persist and/or for as long as the subject continues to test positive for a virus.

In certain embodiments, an infected subject drinks a dose (e.g., an amount of between 10-100 mL, or 20-80 mL, or 30-70 mL) three or four times daily for at least a week, or at least ten days, or at least two weeks, or at least three weeks, or at least a month.

In another embodiment, a person who is not infected (not tested positive for a virus) may be treated with the drinkable formula once or twice daily as a preventative treatment, using the same amounts (volumes) of drinkable formula as an infected subject would consume. For example, a non-infected person may begin a course of treatment if he or she has reason to believe that he or she has come into contact with an infected person.

In certain embodiments, a non-infected person may drink a dose (e.g., an amount of between 10-100 mL, or 20-80 mL, or 30-70 mL) once or twice daily for at least a week, or at least ten days, or at least two weeks, or at least three weeks, or at least a month. A non-infected person may drink the drinkable formula for at least 90 consecutive days or more, 120 consecutive days or more, or even longer.

In certain embodiments, the method of preparing compositions disclosed herein involves mixing the components of the compositions such that, for every “X” total liquid volume of formula, the percentage of RLS to be mixed is to be added at time zero (to) of the production of the formula. Non-limiting examples of the production method are described hereinafter.

The addition of components can be made manually for total volumes less than 20 liters of formula, for example. For larger volumes, addition can be made via a conventional membrane dosifying pump or other comparable pump with flow rates ranging between, for example, 1.5 to 5.0 liters/hour, preferably 2.0 to 3.0 liters/hour.

The infusion of Hydrogas™ is to be made from the time zero (to) of production of the formula. The injection of Hydrogas™, which is conducted for at least 16 minutes and preferably for at least 32 minutes, is preferably carried out via a venturi or recirculating self-priming pump, or other comparable pump.

In an aspect, to secure an efficient injection of Hydrogas™, the power of the self-priming pump needs to be a minimum of 0.1 HP per every 100 liters of formula.

In an aspect, there is no upper limit or unsafe “excess” amount of drinkable formula a subject can consume beyond the limits of hydration and ordinary fluid consumption. In other words, the drinkable formula may be consumed daily, irrespective of infection or symptoms, in the same manner as an individual would consume glasses of water, for example.

A drinkable formula may be prepared by adding:
a. 10-500 mL, preferably 25-350 mL drinking water, preferably distilled or reverse osmosis water; and
b. 0.025 to 15 mL RLS; and
c. mixing Hydrogas™ at a flow rate of 5-500 liters per hour of Hydrogas™, under turbulent conditions, for at least five minutes, preferably at least ten minutes.

In another embodiment, a drinkable formula may be prepared by adding:
a. 50-250 mL, preferably 75-150 mL drinking water, preferably distilled or reverse osmosis water; and
b. 0.1 to 5 mL RLS; and
c. mixing Hydrogas™ at a flow rate of 25-75 liters per hour, preferably 50 liters per hour of Hydrogas™, under turbulent conditions, for at least five minutes, preferably at least ten minutes.

Mouthwash Formula

In another embodiment, the present disclosure provides for compositions prepared using a reducing liquid described herein, prepared as a mouthwash (a “mouthwash formula”).

A mouthwash composition may be prepared by adding:
a. 50-250 mL, preferably 75-150 mL drinking water, preferably distilled or reverse osmosis water; and
b. 0.1 to 5 mL RLS; and
c. mixing Hydrogas™ at a flow rate of 25-75 liters per hour, preferably 50 liters per hour of Hydrogas™, under turbulent conditions, for at least five minutes, preferably at least ten minutes.

In an exemplary method of treating or preventing viral infection, including treating symptoms of viral infection, an individual rinses with the mouthwash formula, swirling or gargling the mouthwash formula once, twice, or three times daily.

In an aspect, an individual may continue treatment using the mouthwash formula for as long as symptoms persist or for as long as he or she is infected with (tests positive for) a virus, or even days, weeks or months after testing negative for a virus.

In an aspect, the mouthwash formula is safe for daily administration, including multiple times daily.

Concentrated Drop Formula

In another embodiment, the present disclosure provides for compositions prepared using a reducing liquid described herein, prepared as a concentrated formula to be applied using a dropper to food or drink (a “concentrated drop formula”).

A concentrated drop formula may be prepared by adding, for every milliliter of total formula volume:
a. 0.1 to 3 mL drinking water, preferably distilled or reverse osmosis water;
b. 0.1 to 3 mL RLS; and
c. mixing Hydrogas™ at a flow rate of 100-500 liters per hour, preferably 250 liters per hour of Hydrogas™, under turbulent conditions, for at least ten minutes, preferably at least fifteen minutes, more preferably at least 20 minutes.

In an exemplary embodiment, mixing is performed for at least 30 minutes.

It has surprisingly been found that the ORP of the resulting concentrated drop formula becomes highly negative, such as −350 mV+/−35 mV.

In an embodiment of the method of treatment using concentrated drops, an individual adds via a dropper 3-12 drops to every liquid he or she drinks throughout the day, preferably 5-10 drops.

In an aspect, the method of treatment using concentrated drops can continue daily for a year or more without interruption. In other embodiments, the treatment period may vary from about 5 days to three years. Also in other embodiments, the oral dose may vary from about 1 drop to about 30 ml, and the dosing frequency may vary from about once daily to about 24 times daily.

In an aspect, the method of treatment using concentrated drops can continue daily indefinitely.

Concentrated Syrup Formula

In a further embodiment, the present disclosure provides for compositions prepared using a reducing liquid described herein, which is a concentrated formula (a “concentrated formula” or “syrup”).

A concentrated syrup of the present invention prepared using a reducing liquid described herein, may be mixed with one or more flavorants or palatants. Inclusion of one or more flavoring agent may aid in subject compliance. Said flavorants or palatants may be one or more natural or artificial flavoring agent. For example and without limitation, compositions described herein may contain honey, ginger, turmeric, matcha powder or other powdered tea, one or more extracts (e.g., vanilla) one or more sugars or sweeteners (e.g., sucrose, fructose, sodium saccharin, sucralose), one or more fruit juices such as alkaline fruit juices, one or more vegetable juices, or other flavoring agents which a skilled person may select.

In certain embodiments, a concentrated syrup formula is prepared using: 300-15000 mL water, preferably distilled or purified water, preferably reverse osmosis (R.O.) water, preferably 500-1000 mL water, preferably distilled or purified water, preferably reverse osmosis (R.O.) water; 50-200 mL RLS, preferably 75-150 mL RLS; 5-60 minutes Hydrogas™ preferably 10-45 minutes Hydrogas™, more preferably 15-30 minutes Hydrogas™; and optionally a flavorant in an amount of 100-500 mL, preferably 200-400 mL.

A concentrated syrup composition may be prepared by adding, for every milliliter of total syrup volume:
a. 0.1 to 3 mL drinking water, preferably distilled or reverse osmosis water;
b. 0.1 to 3 mL RLS; and
c. 0.1 to 0.5 mL, preferably 0.2 mL of an optional flavorant, such as honey or ginger; and

d. mixing Hydrogas™ at a flow rate of 25-75 liters per hour, preferably 50 liters per hour, of Hydrogas™ under turbulent conditions, for at least five minutes, preferably at least ten minutes.

In a first exemplary embodiment, an unflavored concentrated syrup formula is prepared using 900 mL purified water (reverse osmosis or R.O. water), 100 mL RLS, and 20 minutes of the electrolytic process (Hydrogas™) described above.

In a further exemplary embodiment, a honey-flavored concentrated syrup formula is prepared using 600 mL purified water (reverse osmosis or R.O. water), 100 mL RLS, 300 mL honey and 20 minutes of the electrolytic process (Hydrogas™) described above.

In a further exemplary embodiment, a ginger-flavored concentrated syrup formula is prepared using 600 mL purified water (reverse osmosis or R.O. water), 100 mL RLS, 300 mL ginger extract or ginger syrup and 20 minutes of the electrolytic process (Hydrogas™) described above.

The subject drinks the formula daily for, in some cases, a minimum of 3 weeks. In other embodiments, the treatment period may vary from about 10 days to about 180 days. Also in other embodiments, the oral dose may vary from about 5 ml to about 300 ml, and the dosing frequency may vary from about once daily to about 24 times daily.

It has been surprisingly found that in all tested formulations comprising honey and/or ginger root extract, ORP value becomes highly negative, at values of, for example, −840 mV (+/−40 mV).

In one embodiment, a subject infected with a virus (i.e., tested positive) may be treated with about 30 ml of a formula described herein once daily, twice daily, three times daily, four times daily, five times daily, six times daily or more frequently. In a preferred embodiment, the subject is treated once, twice, three times or four times daily.

The subject drinks the formula daily for, in some cases, a minimum of 3 weeks. In other embodiments, the treatment period may vary from about 10 days to about 180 days. Also in other embodiments, the oral dose may vary from about 5 ml to about 300 ml, and the dosing frequency may vary from about once daily to about 24 times daily.

If the person is not infected (tested negative), the person drinks about 30 ml of the formula described herein once a day, in some cases, for at least 90 consecutive days. In other embodiments, the treatment period may vary from about 5 days to about 365 days. Also in other embodiments, the oral dose may vary from about 5 ml to about 300 ml, and the dosing frequency may vary from about once daily to about 24 times daily.

Concentrated syrup formulations according to the present disclosure thus represent an extremely potent antioxidant with no known comparable composition available.

Nasal Drop or Nasal Spray

In a further embodiment, the present disclosure provides for compositions prepared using a reducing liquid described herein, which is a nasal drop or nasal spray formula (a “nasal spray” or “nasal drop” or “nasal formulation”).

A nasal formulation may be prepared by adding:
a. 50-250 mL, preferably 75-150 mL drinking water, preferably distilled or reverse osmosis water; and
b. 0.005 to 1.0 mL RLS, preferably 0.01 to 0.1 mL RLS; and
c. mixing Hydrogas™ at a flow rate of 25-75 liters per hour, preferably 50 liters per hour, more preferably 150 liters per hour of Hydrogas™, under turbulent conditions, for at least five minutes, preferably at least ten minutes.

For every V ml of the mix, mix Hydrogas™ at a flow rate of 150 It of Hydrogas™ per hour, under turbulent conditions, for a time (t) of at least 10 minutes. Note: the ORP becomes negative at (−) 250 mv+/−55 my.

A nasal formulation may be administered either via a dropper as nasal drops, or as a nasal spray via a metered spray device (e.g., nasal inhaler).

In an embodiment of the present method of treatment, at least 3 drops, or 3-10 drops, such as 5-9 drops, such as 6-8 drops in each nostril, once, twice, three times, or four times daily. In a preferred embodiment, three or four doses are administered, each dose being at least 3 drops nostril. In another embodiment, 6-8 drops are administered per nostril per dose.

In another embodiment, of the present method of treatment, a metered spray device is used to administer the nasal formulation as a nasal spray. A person of ordinary skill in the art would readily be capable of adjusting the amount of nasal formula administered per spray to achieve the same amount of dosage as described above with regard to the nasal drop formula.

For example, once a day and/or when the person believes he or she has been in contact with a potentially infected person, the person applies about 3 drops or sprays of the nasal formula described herein to each nostril. In other embodiments, the treatment period may vary from about 5 days to about 365 days. In other embodiments, the treatment period may vary from about 5 days to three years. Also in other embodiments, the oral dose may vary from about 1 drop or spray to about 10 drops or sprays, and the dosing frequency may vary from about once daily to about 24 times daily. When used as a nasal spray, a standard nasal spray dip and tube or atomizer mist delivery device is used, delivering a dose ranging from about 0.05 ml/t to about 1.0 ml/t, where t=the number of sprays applied.

Tablet/Capsule Formulations

In a further embodiment, the present disclosure provides for compositions prepared using a reducing liquid described herein, which is a tablet or a capsule.

In an aspect, a liquid silicon dioxide tablet formulation is described. In a preferred embodiment, a tablet formulation described herein comprises liquid silicon dioxide, microcrystalline cellulose, croscarmellose sodium, and magnesium stearate. A person of ordinary skill in the art will be readily capable of selected amounts and species of the above described components, as well as equivalent different components for inclusion in the tablet formulation.

In an embodiment, the tablet formulation may be prepared by loading microcrystalline cellulose PH 102 in RMG using liquid silicon dioxide. The wet mass is dried at 50 C using a dryer such as a fluid bed dryer until the LOD reaches about 3%. Then the dried granules are milled through a screen, such as a 2 mm screen. The croscarmellose sodium is sifted through mesh, such as #30 mesh and blended with dried granules for at least two minutes, preferably at least three minutes, more preferably for at least five minutes. Then, magnesium stearate is sifted through mesh, such as #40 mesh and blended with dried granules for at least two minutes, preferably at least three minutes, more preferably for at least five minutes. The tablet is then compressed for 600 mg doses. The following Tables 1 and 2 show exemplary, non-limiting parameters for preparing a tablet formulation according to the present invention.

TABLE 1

| Material | Quantity/tablet (mg) | Function |
|---|---|---|
| Liquid silicon dioxide | 494.7 | Active |
| | | substance |
| Microccrystalline cellulose PH 102 | (87.3 | Diluent |
| (Vivapur PH 102) | | |
| Croscarmellose sodium | 12.0 | Disintegrant |
| Magnesium stearate | 6.0 | Lubricant |
| Total | 600.0 | n/a |

TABLE 2

| Parameters | Limit |
|---|---|
| Average weight per | 6.00 +/− 0.3% |
| ten tablets (g) | (5.82 to 6.18)) |
| Weight variation (mg) | 600 +/− 5% |
| | (570.0 to 630.0) |
| DT | NMT 4 minutes |
| Hardness | NLKT 15 Kp |

Additionally, a capsule formulation may be prepared as an alternative to tablets. For example, instead of compressing the formula into a tablet, it may be contained in a capsule.

In an aspect, a tablet or capsule formulation may be administered once, twice, three times or four times daily as needed. In another aspect, any pharmaceutically acceptable carriers, solvents, excipients or other pharmaceutical additives may be incorporated as will be readily understood and selected by a person of ordinary skill in the art.

In an aspect, the present disclosure provides for a method of treating an individual who is susceptible to viral infection, including SARS¬ CoV-2 infection. In another aspect, the present disclosure provides for a method of treating an individual who is infected by a virus, such as SARS¬ CoV-2.

In some embodiments, a composition described herein may be administered at the same time as or in combination with hydroxychloroquine. In other embodiments, a composition described herein may be administered at the same time as or in combination with azithromycin. In still other embodiments, a composition described herein may be administered at the same time as or in combination with hydroxychloroquine and azithromycin.

In some embodiments, a composition described herein may further comprise hydroxychloroquine. In other embodiments, a composition described herein may further comprise azithromycin. In still other embodiments, a composition described herein may further comprise hydroxychloroquine and azithromycin.

In an aspect, any viral infection may be treated with a composition described herein. For example, and without limitation, compositions described herein may be used to treat a patient infected with SARS-CoV-2, influenza, rubella, chickenpox/shingles, roseola, smallpox, viral pneumonia, and the like. Additionally, any symptom of a viral infection may be treated with a composition described herein.

In certain embodiments, a composition described herein is administered to an individual to prevent viral infection. In other embodiments, a composition described herein is administered to an individual to treat viral infection.

In an aspect, the present disclosure provides for a method of ameliorating one or more symptoms resulting from viral infection. In another aspect, the present disclosure provides for a method of treating a viral infection or a disease brought on by viral infection, such as COVID-19.

The detailed description above describes embodiments of a therapeutic composition, a method of preparing the therapeutic composition, and methods of treating a subject involving administration of a composition described herein. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents which fall within the scope of the claims are embraced by the claims.

### EXAMPLES

The present inventors conducted trials to assess the safety and efficacy of the compositions described herein in subjects with coronavirus 2019 (SARS-CoV-2) infection.

The following inclusion criteria were used to select subjects:
a. Male or female adult ≥18 years of age at time of enrollment;
b. Laboratory confirmation of coronavirus 2019 infection by polymerase chain reaction (PCR) or other commercial or public health assay from any diagnostic sampling source;
c. Asymptomatic subjects and/or subjects with mild-to-moderate symptoms of respiratory illness caused by SARS-CoV-2 infection
d. Clinically normal resting 12-lead ECG at Screening Visit or, if abnormal, considered not clinically significant by the Principal Investigator;
e. Subject (or legally authorized representative) provided written informed consent prior to initiation of any procedures; and
f. Understood and agreed to comply with planned study procedures.

The following criteria were used to exclude subjects:
a. Critically ill patients meeting one or more of the following: (1) Experience respiratory failure and need to receive mechanical ventilation; (2) Experience shock; (3) Complicated with other organs failure and need intensive care and therapy in ICU; Unable to take drugs by mouth;
b. Patients with significantly abnormal liver function;
c. Patients in need of dialysis treatment, or GFR ≤30 mL/min/1.73 m²;
d. Participants with severe neurological and mental illness;
e. Pregnant or lactating women;
f. Inability to consent and/or comply with study protocol;
g. Persons already treated with any of the study drugs during the last 30 days.
h. Participants in other clinical trials;
i. Patients with malignant tumors;
j. Co-infection with other infectious viruses or bacteria.

The following Examples summarize the results of a control group and three treatment groups.

### Example 1—Control Group

In a control group, individuals who tested positive for SARS-CoV-2 through a PCR test were administered a saline placebo and hydroxychloroquine. In the control group, none of the individuals exhibited any symptoms.

Nine (9) of the 50 subjects in the control group were resolved from positive to negative through PCR testing after ten days.

A summary of the results of the control group is provided in Table 3 below.

TABLE 3

| | | | | PCR test |
|---|---|---|---|---|
| Pa- | | | | results after |
| tient | Age | Symptoms | Protocol | ten days |
| 1 | 24 | No symptoms | Placebo + | Negative |
| | | | hydroxychloroquine | |
| 2 | 23 | No symptoms | Placebo + | Negative |
| | | | hydroxychloroquine | |
| 3 | 38 | No symptoms | Placebo + | Negative |
| | | | hydroxychloroquine | |
| 4 | 22 | No symptoms | Placebo + | Negative |
| | | | hydroxychloroquine | |
| 5 | 45 | No symptoms | Placebo + | Negative |
| | | | hydroxychloroquine | |
| 6 | Not available | No symptoms | Placebo + | Positive |
| | | | hydroxychloroquine | |
| 7 | Not available | No symptoms | Placebo + | Positive |
| | | | hydroxychloroquine | |
| 8 | Not available | No symptoms | Placebo + | Positive |
| | | | hydroxychloroquine | |
| 9 | Not available | No symptoms | Placebo + | Positive |
| | | | hydroxychloroquine | |
| 10 | Not available | No symptoms | Placebo + | Positive |
| | | | hydroxychloroquine | |
| 11 | Not available | No symptoms | Placebo + | Positive |
| | | | hydroxychloroquine | |
| 12 | 32 | No symptoms | Placebo + | Negative |
| | | | hydroxychloroquine | |
| 13 | 34 | No symptoms | Placebo + | Negative |
| | | | hydroxychloroquine | |
| 14 | 34 | No symptoms | Placebo + | Negative |
| | | | hydroxychloroquine | |
| 15 | 20 | No symptoms | Placebo + | Negative |
| | | | hydroxychloroquine | |
| 16 | Not available | No symptoms | Placebo + | Positive |
| | | | hydroxychloroquine | |
| 17 | Not available | No symptoms | Placebo + | Positive |
| | | | hydroxychloroquine | |
| 18 | Not available | No symptoms | Placebo + | Positive |
| | | | hydroxychloroquine | |
| 19 | Not available | No symptoms | Placebo + | Positive |
| | | | hydroxychloroquine | |
| 20 | 28 | No symptoms | Placebo + | Positive |
| | | | hydroxychloroquine | |
| 21 | 21 | No symptoms | Placebo + | Positive |
| | | | hydroxychloroquine | |
| 22 | Not available | No symptoms | Placebo + | Positive |
| | | | hydroxychloroquine | |
| 23 | Not available | No symptoms | Placebo + | Positive |
| | | | hydroxychloroquine | |
| 24 | 30 | No symptoms | Placebo + | Positive |
| | | | hydroxychloroquine | |
| 25 | 34 | No symptoms | Placebo + | Positive |
| | | | hydroxychloroquine | |
| 26 | Not available | No symptoms | Placebo + | Positive |
| | | | hydroxychloroquine | |
| 27 | 42 | No symptoms | Placebo + | Positive |
| | | | hydroxychloroquine | |
| 28 | 29 | No symptoms | Placebo + | Positive |
| | | | hydroxychloroquine | |
| 29 | 31 | No symptoms | Placebo + | Positive |
| | | | hydroxychloroquine | |
| 30 | 29 | No symptoms | Placebo + | Positive |
| | | | hydroxychloroquine | |
| 31 | 24 | No symptoms | Placebo + | Positive |
| | | | hydroxychloroquine | |
| 32 | 22 | No symptoms | Placebo + | Positive |
| | | | hydroxychloroquine | |
| 33 | 22 | No symptoms | Placebo + | Positive |
| | | | hydroxychloroquine | |
| 34 | 22 | No symptoms | Placebo + | Positive |
| | | | hydroxychloroquine | |
| 35 | 21 | No symptoms | Placebo + | Positive |
| | | | hydroxychloroquine | |
| 36 | 43 | No symptoms | Placebo + | Positive |
| | | | hydroxychloroquine | |
| 37 | 21 | No symptoms | Placebo + | Positive |
| | | | hydroxychloroquine | |
| 38 | 26 | No symptoms | Placebo + | Positive |
| | | | hydroxychloroquine | |
| 39 | 22 | No symptoms | Placebo + | Positive |
| | | | hydroxychloroquine | |
| 40 | 29 | No symptoms | Placebo + | Positive |
| | | | hydroxychloroquine | |
| 41 | 40 | No symptoms | Placebo + | Positive |
| | | | hydroxychloroquine | |
| 42 | 20 | No symptoms | Placebo + | Positive |
| | | | hydroxychloroquine | |
| 43 | 41 | No symptoms | Placebo + | Positive |
| | | | hydroxychloroquine | |
| 44 | 27 | No symptoms | Placebo + | Positive |
| | | | hydroxychloroquine | |
| 45 | 33 | No symptoms | Placebo + | Positive |
| | | | hydroxychloroquine | |
| 46 | 43 | No symptoms | Placebo + | Positive |
| | | | hydroxychloroquine | |
| 47 | 23 | No symptoms | Placebo + | Positive |
| | | | hydroxychloroquine | |
| 48 | 44 | No symptoms | Placebo + | Positive |
| | | | hydroxychloroquine | |
| 49 | 45 | No symptoms | Placebo + | Positive |
| | | | hydroxychloroquine | |
| 50 | 34 | No symptoms | Placebo + | Positive |
| | | | hydroxychloroquine | |

### Example 2—Treatment Group I

In a first treatment group (Treatment Group I), 50 symptomatic individuals who tested positive for SARS-CoV-2 through a PCR test were administered drinking formula, concentrated (syrup) formula and nasal formula.

Drinking formula administered: 60 mL four times daily (every six hours)

Concentrated (syrup) formula administered: 8 drops added to any liquid patients drank throughout each day

Nasal formula administered: 6-8 drops applied in each nostril four times daily (every six hours)

35 of the 50 subjects in the control group were resolved from positive to negative through PCR testing after ten days.

A summary of the results of Treatment Group I is provided in Table 4 below.

TABLE 4

| | | | | PCR test |
|---|---|---|---|---|
| | | | | results |
| Pa- | | | | after |
| tient | Age | Symptoms | Protocol | ten days |
| 1 | 32 | Bodyache | Drinking formula, | Negative |
| | | | concentrated syrup, | |
| | | | nasal formula | |
| 2 | 33 | Cough, fever | Drinking formula, | Negative |
| | | | concentrated syrup, | |
| | | | nasal formula | |
| 3 | 30 | Breathing difficulties, | Drinking formula, | Negative |
| | | cough | concentrated syrup, | |
| | | | nasal formula | |
| 4 | 30 | Chest pain, cough | Drinking formula, | Negative |
| | | | concentrated syrup, | |
| | | | nasal formula | |
| 5 | 30 | Severe fever, bodyache | Drinking formula, | Negative |
| | | | concentrated syrup, | |
| | | | nasal formula | |
| 6 | 30 | Fever, cold | Drinking formula, | Negative |
| | | | concentrated syrup, | |
| | | | nasal formula | |
| 7 | 43 | Nasal congestion, | Drinking formula, | Negative |
| | | cough | concentrated syrup, | |
| | | | nasal formula | |
| 8 | 21 | Throat pain | Drinking formula, | Negative |
| | | | concentrated syrup, | |
| | | | nasal formula | |
| 9 | 32 | Mild headache | Drinking formula, | Negative |
| | | | concentrated syrup, | |
| | | | nasal formula | |
| 10 | 28 | Cold, cough, fever | Drinking formula, | Negative |
| | | | concentrated syrup, | |
| | | | nasal formula | |
| 11 | 24 | Throat pain, fever | Drinking formula, | Negative |
| | | | concentrated syrup, | |
| | | | nasal formula | |
| 12 | 24 | Cough, fever | Drinking formula, | Negative |
| | | | concentrated syrup, | |
| | | | nasal formula | |
| 13 | 25 | Nasal congestion | Drinking formula, | Negative |
| | | | concentrated syrup, | |
| | | | nasal formula | |
| 14 | 26 | Nasal congestion | Drinking formula, | Negative |
| | | | concentrated syrup, | |
| | | | nasal formula | |
| 15 | 27 | Nasal congestion, | Drinking formula, | Negative |
| | | cough | concentrated syrup, | |
| | | | nasal formula | |
| 16 | 23 | Cough, fever | Drinking formula, | Negative |
| | | | concentrated syrup, | |
| | | | nasal formula | |
| 17 | 34 | Chest pain, cough | Drinking formula, | Negative |
| | | | concentrated syrup, | |
| | | | nasal formula | |
| 18 | 43 | Throat pain | Drinking formula, | Negative |
| | | | concentrated syrup, | |
| | | | nasal formula | |
| 19 | 43 | Mild headache | Drinking formula, | Negative |
| | | | concentrated syrup, | |
| | | | nasal formula | |
| 20 | 40 | Cough, headache | Drinking formula, | Negative |
| | | | concentrated syrup, | |
| | | | nasal formula | |
| 21 | 22 | Fever | Drinking formula, | Negative |
| | | | concentrated syrup, | |
| | | | nasal formula | |
| 22 | 21 | Fever | Drinking formula, | Negative |
| | | | concentrated syrup, | |
| | | | nasal formula | |
| 23 | 21 | Breathing difficulties | Drinking formula, | Negative |
| | | | concentrated syrup, | |
| | | | nasal formula | |
| 24 | 21 | Chest pain, cough | Drinking formula, | Negative |
| | | | concentrated syrup, | |
| | | | nasal formula | |
| 25 | 25 | Chest pain, cough | Drinking formula, | Negative |
| | | | concentrated syrup, | |
| | | | nasal formula | |
| 26 | 22 | Chest pain, cough | Drinking formula, | Negative |
| | | | concentrated syrup, | |
| | | | nasal formula | |
| 27 | 23 | Mild headache | Drinking formula, | Negative |
| | | | concentrated syrup, | |
| | | | nasal formula | |
| 28 | 36 | Cough, fever | Drinking formula, | Negative |
| | | | concentrated syrup, | |
| | | | nasal formula | |
| 29 | 39 | Cough, headache | Drinking formula, | Negative |
| | | | concentrated syrup, | |
| | | | nasal formula | |
| 30 | 38 | Cough, headache | Drinking formula, | Negative |
| | | | concentrated syrup, | |
| | | | nasal formula | |
| 31 | 39 | Cough | Drinking formula, | Negative |
| | | | concentrated syrup, | |
| | | | nasal formula | |
| 32 | 38 | Fever, rashes on skin | Drinking formula, | Negative |
| | | | concentrated syrup, | |
| | | | nasal formula | |
| 33 | 30 | Headache, fever | Drinking formula, | Negative |
| | | | concentrated syrup, | |
| | | | nasal formula | |
| 34 | 43 | Cold, cough, fever | Drinking formula, | Negative |
| | | | concentrated syrup, | |
| | | | nasal formula | |
| 35 | 21 | Cold, cough, fever | Drinking formula, | Negative |
| | | | concentrated syrup, | |
| | | | nasal formula | |
| 36 | 34 | Cold, cough, fever | Drinking formula, | Positive |
| | | | concentrated syrup, | |
| | | | nasal formula | |
| 37 | 36 | Chest pain, cough | Drinking formula, | Positive |
| | | | concentrated syrup, | |
| | | | nasal formula | |
| 38 | 26 | Breathing difficulties | Drinking formula, | Positive |
| | | | concentrated syrup, | |
| | | | nasal formula | |
| 39 | 20 | Breathing difficulties, | Drinking formula, | Positive |
| | | cough | concentrated syrup, | |
| | | | nasal formula | |
| 40 | 29 | Fever | Drinking formula, | Positive |
| | | | concentrated syrup, | |
| | | | nasal formula | |
| 41 | 37 | Fever, cold | Drinking formula, | Positive |
| | | | concentrated syrup, | |
| | | | nasal formula | |
| 42 | 39 | Chest pain, cough | Drinking formula, | Positive |
| | | | concentrated syrup, | |
| | | | nasal formula | |
| 43 | 38 | Fever, cough | Drinking formula, | Positive |
| | | | concentrated syrup, | |
| | | | nasal formula | |
| 44 | 20 | Fever, cough | Drinking formula, | Positive |
| | | | concentrated syrup, | |
| | | | nasal formula | |
| 45 | 22 | Breathing difficulties, | Drinking formula, | Positive |
| | | cough | concentrated syrup, | |
| | | | nasal formula | |
| 46 | 28 | Chest pain, cough | Drinking formula, | Positive |
| | | | concentrated syrup, | |
| | | | nasal formula | |
| 47 | 27 | Chest pain, cough | Drinking formula, | Positive |
| | | | concentrated syrup, | |
| | | | nasal formula | |
| 48 | 34 | Rashes on skin, | Drinking formula, | Positive |
| | | headache and cough | concentrated syrup, | |
| | | | nasal formula | |
| 49 | 34 | Breathing difficulties, | Drinking formula, | Positive |
| | | cough | concentrated syrup, | |
| | | | nasal formula | |
| 50 | 45 | Breathing difficulties, | Drinking formula, | Positive |
| | | cough | concentrated syrup, | |
| | | | nasal formula | |

### Example 3—Treatment Group II

In a second treatment group (Treatment Group II), 30 symptomatic individuals who tested positive for SARS-CoV-2 through a PCR test were administered drinking formula, concentrated (syrup) formula and nasal formula, as well as hydroxychloroquine (three 100 mg doses per day for six days) and azithromycin (500 mg per day for three days). In the control group, none of the individuals exhibited any symptoms.

Drinking formula administered: 60 mL four times daily (every six hours)

Concentrated (syrup) formula administered: 8 drops added to any liquid patients drank throughout each day

Nasal formula administered: 6-8 drops applied in each nostril four times daily (every six hours)

All 30 subjects in Treatment Group II were resolved from positive to negative through PCR testing after ten days.

A summary of the results of Treatment Group II is provided in Table 5 below.

TABLE 5

| | | | | PCR test |
|---|---|---|---|---|
| | | | | results |
| Pa- | | | | after |
| tient | Age | Symptoms | Protocol | ten days |
| 1 | 24 | Cough, fever | Drinking formula, | Negative |
| | | | concentrated syrup, nasal | |
| | | | formula, hydroxychloroquine, | |
| | | | azithromycin | |
| 2 | 38 | Chest pain, | Drinking formula, | Negative |
| | | discomfort in | concentrated syrup, nasal | |
| | | breathing | formula, hydroxychloroquine, | |
| | | | azithromycin | |
| 3 | 36 | Fever | Drinking formula, | Negative |
| | | | concentrated syrup, nasal | |
| | | | formula, hydroxychloroquine, | |
| | | | azithromycin | |
| 4 | 33 | Vomiting, | Drinking formula, | Negative |
| | | fever | concentrated syrup, nasal | |
| | | | formula, hydroxychloroquine, | |
| | | | azithromycin | |
| 5 | 34 | Bodyache, | Drinking formula, | Negative |
| | | fever, cough | concentrated syrup, nasal | |
| | | | formula, hydroxychloroquine, | |
| | | | azithromycin | |
| 6 | 22 | Breathing | Drinking formula, | Negative |
| | | difficulties | concentrated syrup, nasal | |
| | | | formula, hydroxychloroquine, | |
| | | | azithromycin | |
| 7 | 33 | Bodyache | Drinking formula, | Negative |
| | | | concentrated syrup, nasal | |
| | | | formula, hydroxychloroquine, | |
| | | | azithromycin | |
| 8 | 25 | Fever, cough | Drinking formula, | Negative |
| | | | concentrated syrup, nasal | |
| | | | formula, hydroxychloroquine, | |
| | | | azithromycin | |
| 9 | 24 | Fever | Drinking formula, | Negative |
| | | | concentrated syrup, nasal | |
| | | | formula, hydroxychloroquine, | |
| | | | azithromycin | |
| 10 | 33 | Cough, | Drinking formula, | Negative |
| | | breathing | concentrated syrup, nasal | |
| | | problems | formula, hydroxychloroquine, | |
| | | | azithromycin | |
| 11 | 45 | Diabetic, | Drinking formula, | Negative |
| | | loss of | concentrated syrup, nasal | |
| | | appetite, fever, | formula, hydroxychloroquine, | |
| | | weakness | azithromycin | |
| 12 | 22 | Cough, fever | Drinking formula, | Negative |
| | | | concentrated syrup, nasal | |
| | | | formula, hydroxychloroquine, | |
| | | | azithromycin | |
| 13 | 42 | Fever | Drinking formula, | Negative |
| | | | concentrated syrup, nasal | |
| | | | formula, hydroxychloroquine, | |
| | | | azithromycin | |
| 14 | 20 | Breathing | Drinking formula, | Negative |
| | | difficulties | concentrated syrup, nasal | |
| | | | formula, hydroxychloroquine, | |
| | | | azithromycin | |
| 15 | 45 | Bodyache, | Drinking formula, | Negative |
| | | fever, cough | concentrated syrup, nasal | |
| | | | formula, hydroxychloroquine, | |
| | | | azithromycin | |
| 16 | 24 | Bodyache, | Drinking formula, | Negative |
| | | fever, cough | concentrated syrup, nasal | |
| | | | formula, hydroxychloroquine, | |
| | | | azithromycin | |
| 17 | 23 | Fever, cold, | Drinking formula, | Negative |
| | | cough | concentrated syrup, nasal | |
| | | | formula, hydroxychloroquine, | |
| | | | azithromycin | |
| 18 | 23 | Cough | Drinking formula, | Negative |
| | | | concentrated syrup, nasal | |
| | | | formula, hydroxychloroquine, | |
| | | | azithromycin | |
| 19 | 22 | Breathing | Drinking formula, | Negative |
| | | difficulties | concentrated syrup, nasal | |
| | | | formula, hydroxychloroquine, | |
| | | | azithromycin | |
| 20 | 22 | Breathing | Drinking formula, | Negative |
| | | difficulties, | concentrated syrup, nasal | |
| | | fever | formula, hydroxychloroquine, | |
| | | | azithromycin | |
| 21 | 33 | Weakness, | Drinking formula, | Negative |
| | | bodyache | concentrated syrup, nasal | |
| | | | formula, hydroxychloroquine, | |
| | | | azithromycin | |
| 22 | 34 | Fever, cough | Drinking formula, | Negative |
| | | | concentrated syrup, nasal | |
| | | | formula, hydroxychloroquine, | |
| | | | azithromycin | |
| 23 | 42 | Cough | Drinking formula, | Negative |
| | | | concentrated syrup, nasal | |
| | | | formula, hydroxychloroquine, | |
| | | | azithromycin | |
| 24 | 20 | Fever, cold, | Drinking formula, | Negative |
| | | cough | concentrated syrup, nasal | |
| | | | formula, hydroxychloroquine, | |
| | | | azithromycin | |
| 25 | 33 | Chest pain, | Drinking formula, | Negative |
| | | discomfort in | concentrated syrup, nasal | |
| | | breathing | formula, hydroxychloroquine, | |
| | | | azithromycin | |
| 26 | 28 | Throatache, | Drinking formula, | Negative |
| | | fever | concentrated syrup, nasal | |
| | | | formula, hydroxychloroquine, | |
| | | | azithromycin | |
| 27 | 42 | Breathing | Drinking formula, | Negative |
| | | difficulties | concentrated syrup, nasal | |
| | | | formula, hydroxychloroquine, | |
| | | | azithromycin | |
| 28 | 22 | Bodyache, | Drinking formula, | Negative |
| | | fever, | concentrated syrup, nasal | |
| | | cough | formula, hydroxychloroquine, | |
| | | | azithromycin | |
| 29 | 37 | Fever, cough | Drinking formula, | Negative |
| | | | concentrated syrup, nasal | |
| | | | formula, hydroxychloroquine, | |
| | | | azithromycin | |
| 30 | 45 | Fever, cough | Drinking formula, | Negative |
| | | | concentrated syrup, nasal | |
| | | | formula, hydroxychloroquine, | |
| | | | azithromycin | |

### Example 4—Treatment Group III

In a second treatment group (Treatment Group III), 50 asymptomatic or mildly symptomatic individuals who tested positive for SARS-CoV-2 through a PCR test were administered drinking formula.

Drinking formula administered: 60 mL four times daily (every six hours)

All 50 subjects in Treatment Group III were resolved from positive to negative through PCR testing after ten days.

A summary of the results of Treatment Group III is provided in Table 6 below.

TABLE 6

| | | | | PCR test |
|---|---|---|---|---|
| | | | | results |
| | | | | after |
| Patient | Age | Symptoms | Protocol | ten days |
| 1 | 38 | Asymptomatic | Drinking formula | Negative |
| 2 | 44 | Asymptomatic | Drinking formula | Negative |
| 3 | 22 | Mild cough | Drinking formula | Negative |
| 4 | 21 | Fever, cough | Drinking formula | Negative |
| 5 | 43 | Asymptomatic | Drinking formula | Negative |
| 6 | 32 | Asymptomatic | Drinking formula | Negative |
| 7 | 33 | Asymptomatic | Drinking formula | Negative |
| 8 | 40 | Cough | Drinking formula | Negative |
| 9 | 23 | Asymptomatic | Drinking formula | Negative |
| 10 | 3939 | Mild cough | Drinking formula | Negative |
| 11 | 38 | Asymptomatic | Drinking formula | Negative |
| 12 | 20 | Asymptomatic | Drinking formula | Negative |
| 13 | 32 | Mild bodyache | Drinking formula | Negative |
| 14 | 21 | Asymptomatic | Drinking formula | Negative |
| 15 | 40 | Asymptomatic | Drinking formula | Negative |
| 16 | 39 | Asymptomatic | Drinking formula | Negative |
| 17 | 20 | Asymptomatic | Drinking formula | Negative |
| 18 | 31 | Mild fever | Drinking formula | Negative |
| 19 | 25 | Asymptomatic | Drinking formula | Negative |
| 20 | 36 | Mild fever | Drinking formula | Negative |
| 21 | 30 | Mild fever | Drinking formula | Negative |
| 22 | 32 | Mild cough | Drinking formula | Negative |
| 23 | 22 | Asymptomatic | Drinking formula | Negative |
| 24 | 39 | Asymptomatic | Drinking formula | Negative |
| 25 | 44 | Asymptomatic | Drinking formula | Negative |
| 26 | 24 | Mild fever | Drinking formula | Negative |
| 27 | 44 | Mild cough | Drinking formula | Negative |
| 28 | 43 | Asymptomatic | Drinking formula | Negative |
| 29 | 40 | Mild fever | Drinking formula | Negative |
| 30 | 28 | Asymptomatic | Drinking formula | Negative |
| 31 | 27 | Asymptomatic | Drinking formula | Negative |
| 32 | 34 | Asymptomatic | Drinking formula | Negative |
| 33 | 33 | Asymptomatic | Drinking formula | Negative |
| 34 | 21 | Asymptomatic | Drinking formula | Negative |
| 35 | 43 | Mild fever | Drinking formula | Negative |
| 36 | 45 | Asymptomatic | Drinking formula | Negative |
| 37 | 33 | Asymptomatic | Drinking formula | Negative |
| 38 | 35 | Asymptomatic | Drinking formula | Negative |
| 39 | 29 | Asymptomatic | Drinking formula | Negative |
| 40 | 45 | Asymptomatic | Drinking formula | Negative |
| 41 | 42 | Mild cough | Drinking formula | Negative |
| 42 | 40 | Mild cough | Drinking formula | Negative |
| 43 | 38 | Asymptomatic | Drinking formula | Negative |
| 44 | 36 | Asymptomatic | Drinking formula | Negative |
| 45 | 33 | Mild bodyache | Drinking formula | Negative |
| 46 | 43 | Asymptomatic | Drinking formula | Negative |
| 47 | 44 | Asymptomatic | Drinking formula | Negative |
| 48 | 37 | Asymptomatic | Drinking formula | Negative |
| 49 | 38 | Asymptomatic | Drinking formula | Negative |
| 50 | 42 | Asymptomatic | Drinking formula | Negative |

### REFERENCES

[1] Akkaya A, Öztürk Ö. Total antioxidant capacity and C-reactive protein levels in patients with community-acquired pneumonia. Turk J Medicalences 2008; 38:537-44.
[2] Du, Ting & Liang, Jiangong & Lu, Jian & Fu, Yiying & Fang, Liurong & Xiao, Shaobo & Han, He-You. (2018). Glutathione-Capped Ag2S Nanoclusters Inhibit Coronavirus Proliferation through Blockage of Viral RNA Synthesis and Budding. ACS Applied Materials & Interfaces. 10. 10.1021/acsami.7b13811.
[3] Food and Drug Administration (FDA) Guidance on Conduct of Clinical Trials of Medical Products during COVID-19 Pandemic. U.S. Department of Health and Human Services Food and Drug Administration. March 2020.
[4] Qianwen Zhang, Yuanrong Ju, Yan Ma, Tao Wang. N-acetylcysteine improves oxidative stress and inflammatory response in patients with community acquired pneumonia. Medicine (Baltimore). 2018 November; 97(45): e13087.
[5] Townsend D, White L M, Lester C E, DeLeon R C, Cisneros, I, Maitin V, Richardson C R, Vattem, D A. Evaluation of Potential Anti-Pathogenic and Anti-Retroviral Effects of a Proprietary Bioactive Silicate Alka-VitaTM/Alka-V6TM/AlkahydroxyTM (AVAH). International Journal of Applied Research in Natural Products Vol. 3 (4), pp. 19-28, December 2010-January 2011.
[6] Townsend D, DeLeon R C, Lester C E, White L M, Maitin V, Cisneros, I, Richardson C R, Vattem, D A. Evaluation of Potential Redox Modulatory and Chemotherapeutic Effects of a Proprietary Bioactive Silicate Alka-VitaTM/Alka-V6TM/AlkahydroxyTM (AVAH). International Journal of Applied Research in Natural Products Vol. 3 (4), pp. 5-18, December 2010-January 2011.
[7] Valko M, Leibfritz D, Moncol J, et al. Free radicals and antioxidants in normal physiological functions and human disease. Int J Biochem Cell Biol 2007; 39:44-84.
[8] Zimmermann, Petra & Curtis, Nigel. Coronavirus Infections in Children Including COVID-19. An Overview of the Epidemiology, Clinical Features, Diagnosis, Treatment and Prevention Options in Children. The Pediatric Infectious Disease Journal: Mar. 12, 2020.

1. A method of treating or preventing a viral infection or a symptom thereof comprising administering to an individual in need thereof a composition comprising an aqueous solution infused with a metasilicate and reducing gas, wherein the composition has an oxidation reduction potential (ORP) value of about −100 mV or more negative.

2. The method of claim 1, wherein the composition is prepared by a process comprising:
infusing the aqueous solution with a reducing gas and a metasilicate, wherein the infusing involves mixing under turbulent conditions, and
wherein the reducing gas and/or the metasilicate reacts with the aqueous solution to produce a reducing liquid having the oxidation reduction potential (ORP) value of about −100 mV or more negative.

3. The method of claim 2, wherein the mixing occurs at a flow rate of 100-500 liters of reducing gas per hour under turbulent conditions, for at least five minutes.

4. The method of claim 2, wherein the mixing occurs at a flow rate of 25-75 liters of reducing gas per hour under turbulent conditions for at least five minutes.

5. The method of claim 1, wherein the composition is a drinkable formulation administered in an amount of 10-100 mL three or four times daily for at least a week.

6. The method of claim 1, wherein the composition is a mouthwash formula administered by rinsing or gargling, the mouthwash formula prepared by adding, per milliliter of total mouthwash formula volume, 50-250 mL water and 0.1 to 5 mL metasilicate, and mixing the water and metasilicate with reducing gas at a flow rate of 25-75 liters of reducing gas per hour under turbulent conditions for at least five minutes.

7. The method of claim 1, wherein the composition is formulated for nasal administration;
wherein 0.025 mL to 0.50 mL composition is administered per nostril once, twice, three times, four times, five times, or six times a day; and
wherein the composition is administered via an atomizer mist delivery device configured to deliver a dose ranging from about 0.05 ml/t to about 1.0 ml/t, where “t” is the number of sprays applied per dose.

8. The method of claim 1, wherein the composition is formulated as a concentrated syrup prepared by mixing the aqueous solution, metasilicate and reducing gas at a flow rate of 25-75 liters of reducing gas per hour under turbulent conditions, for at least five minutes, wherein the individual drinks 30 mL concentrated syrup once daily, twice daily, three times daily, four times daily, five times daily, or six times daily.

9. The method of claim 8, wherein the individual has not tested positive for a viral infection and wherein the individual drinks 30 mL concentrated syrup once daily.

10. The method of claim 8, wherein the individual has tested positive for a viral infection and wherein the individual drinks 30 mL concentrated syrup three or four times daily.

11. The method of claim 8, wherein the composition further comprises a flavorant selected from honey and ginger root extract and wherein the composition has an ORP value of −500 mV or more negative.

12. The method of claim 1, wherein the virus is SARS-CoV-2 or influenza.

13. The method of claim 12, wherein the composition is administered in combination with hydroxychloroquine and/or azithromycin.

14. A composition for preventing or treating viral infection or symptoms thereof, comprising an aqueous solution infused with a metasilicate and reducing gas, wherein the ORP value of the composition is −100 mV or more negative.

15. The composition of claim 14, wherein the metasilicate is a sodium silicate complex with a pH of 13.7.

16. The composition of claim 14, further comprising honey or ginger root extract.

17. The composition claim 14, wherein the ORP value of the composition is −300 mV or more negative.

18. The composition claim 14, wherein the ORP value of the composition is −500 mV or more negative.

19. The composition of claim 14, further comprising hydroxychloroquine.

20. The composition of claim 14, further comprising azithromycin.
